(19) United States
(12) Patent Application Publication
(10) Pub. No.: US 2008/0235881 A1
(43) Pub. Date: Oct. 2, 2008

(54) Method and Device for the Direct Mixing of Chemicals, Especially for Producing Hair Dyes and/or Intensive Toners

(76) Inventor: Vera Lauper, Neuheim (CH)

Correspondence Address: NIXON PEABODY, LLP, 401 9TH STREET, NW, SUITE 900, WASHINGTON, DC, 20004-2128, US

(21) Appl. No.: 10/587,397

(22) PCT Filed: Jan. 20, 2005
(86) PCT No.: PCT/DE05/00077

§ 371 Date: Dec. 6, 2007

(30) Foreign Application Priority Data
Jan. 25, 2004 (DE) 10 2004 004 258.6

(51) Int. Cl. A61K 8/18; A61Q 5/10; B65D 25/08

(52) U.S. Cl. 8/408; 206/219

(57) ABSTRACT

The invention relates to a method for the production of hair dyes and/or intensive toners, whereby the intermediates (developer and coupler) and the direct dye are dissolved in liquid or creamy support masses, which may be combined in a computer-controlled device in situ, based on formulations stored in the computer to give a colour tone mixture, for example, in the hairdressers, or in a shop. According to the multi-step method, the applied intermediates (developer and coupler) and the direct dye are dissolved in a support mass in an initial step, whereby only sufficient alkali agent is added to dissolve and stabilise the intermediates (developer and coupler), the direct dye and the support mass. In a subsequent further method step, after mixing of the individual base components, generated in the initial stage, optionally ammonia or corresponding substitute materials are added before the addition of the peroxide.

### TECHNICAL FIELD

The invention relates to a method for producing hair dyes and/or intensive toners in which the intermediates (developer and coupler) and the direct dyes are dissolved in liquid or creamy carrier masses that are mixed together by a computer-controlled device based on recipes stored in the computer to a tone mixture on site, e.g., in a hairdressing salon or in a shop.

### PRIOR ART

German application 101 14 060 A1 teaches a method and a device with which classic oxidation hair dyes and also intensive toners can be produced given appropriate equipping of the storage device with base components and appropriate recipes. The intermediates (developer and coupler), required for creating the tone, and the direct dyes are dissolved individually here together with other chemicals in a carrier mass and filled as so-called base components into flexible bags. The bags are placed in magazines fastened, e.g., on a turntable, and connected via connectors to pumps (e.g., piston pumps). The tone is composed via a computer in which the recipes and/or the composition of the desired tones by mixing the individual base components are stored, by fetching the recipe for the particular hair color desired. This type of ready-to-use production of hair dyes and/or intensive toners has the advantage that many tones can be produced on site, that is, e.g., in a hairdressing salon or in a shop, with few base components. As is described in German application 101 14 060 A1, the base components filled in the bags yield the ready-to-use hair dye after the mixing process.

Only the peroxide needs to be added prior to use. Thus, only the production of classic, ammonia-containing hair dyes is possible with the method and the device used for this method that are known from said application, in which only the peroxide needs to be added prior to use. Of course, such a device can also be used to produce physical toners to the extent that other, appropriately designed base components are added.

As is known, oxidation hair dyes are generally composed of intermediates (developers and couplers) and direct dyes dissolved in a stable manner in an ammonia-containing carrier mass.

The carrier mass used can be present in liquid or creamy form such as described, e.g., in the “Handbuch der Kosmetika und Riechstoffe” [Handbook of Cosmetics and Perfumes] published by the A. Hütig Verlag, Heidelberg, 2^{nd} edition, volume 3, or in “Grundlagen für klare, flüssige Haarfarben” [Bases for Clear, Liquid Hair Dyes] by Hugo Janistin. Other examples can also be found in “The Chemistry and Manufacture of Cosmetics” by Maison G. de Navarre, volume IV, published by Allured Publishing Corp., Illinois, USA, 3^{rd} edition, or in “Grundlagen und Rezepturen der Kosmetik” [Bases and Recipes of Cosmetics] by Karlheinz Schrader published by the A. Hütig Verlag, Heidelberg, 2^{nd} edition.

The appropriate intermediates (developer and coupler) and direct dyes as well as one or more alkalizing agents are added to these carrier masses or to the particular carrier mass selected. The finished product produced in this manner, the oxidation hair dye, is subsequently filled into containers such as tubes or bottles. The oxidation hair dye is mixed with 1 to 3 parts of a peroxide solution immediately before use and applied onto the hair to be dyed.

Classic oxidation hair dyes contain ammonia in order to simultaneously achieve a certain brightening effect of the natural hair during the dying process; on the other hand, intensive toners contain only little or no ammonia, but contain other alkalizing agents such as sodium hydroxide, ethanolamine, etc. If the hairdresser wants to offer both types of oxidation dyes he must therefore have a double assortment. It is the object of the present invention to give the hairdresser or customer in the shop the possibility of being able to selectively produce classic hair dyes or intensive toners on site with the same base components.

The solution of the object underlying the invention is indicated in claim 1, which describes a method according to the invention.

The subject matter of patent claim 6 is a device for carrying out the method.

As a result of this method only as much alkalizing agent (e.g., sodium hydroxide) is added to the base components in a precursor stage as is necessary for the dissolving and the stabilizing of the intermediates (developer and coupler), the direct dyes and the carrier mass. For completion, other alkalizing agents, such as ammonia and/or ethanolamine and/or sodium hydroxide, worked into the carrier mass must be added in a further stage depending on the desired type of coloring (classic hair dyes or intensive toners). For dyeing or toning, the particular alkalizing agent desired or required is added to the dye mass immediately before use and the entirety is mixed as customarily in a further step with peroxide and is applied.

The advantage of this procedure is that the hairdresser or a competent attendant in a shop can produce the classic ammonia-containing oxidation hair dye as well as the ammonia-free intensive toner himself, e.g., immediately before use, with the same base components by selecting the desired alkalizing agent in the following stage.

Customary alkalizing agents can be used as alkalizing agents in the second stage.

It has proven to be advantageous in the production of the base components to combine the separate aqueous phases and fatty phases at 70 to 80° C. and to mix in the treatment substances and perfume substances at approximately 50° C. The filling of the ready base components into flexible bags takes place in the ideal instance at 45 to 40° C.

In order to be able to carry out the production of the classic hair dye or intensive toner on site, that is, in the salon or in a shop, a device has proven to be advantageous in which the bags with the base components can be suspended or placed and which comprises control means for filling the masses into a container. The control of the device is designed in such a manner that at first the base components are combined and subsequently alkalizing agents are added in a further step during which the hairdresser can decide between the various alkalizing agents, depending on the type of dyeing. The peroxide is subsequently added only after the conclusion of the first two stages or, if the hair dye or intensive toner is not to be used in a salon, is placed in a container, e.g., a bottle. The hairdresser and the customer, respectively, have the advantage, due to the possibility of selecting the alkalizing agents, of being able to produce both classic, ammonia-containing hair dyes as well as intensive toners in the salon or in the shop with the same device and by using the same base components.

In order to be able to meet all requirements such as, e.g., the shading of the tone, the brightness of the tone, etc., the base components, the alkalizing agents and the peroxides are held available in various concentrations in the device.

Brief description of the invention and ways of carrying out the invention; industrial usability

An exemplary embodiment of the invention is described in detail in the following. The device for producing hair dyes and intensive toners consists as is described in German application 101 14 060 A1 of a motor-driven turntable on which, e.g., magazines or containers for depositing flexible bags are fastened. Each of these bags is connected, e.g., by a connector and a control valve to a pump (e.g., piston pump). The pump exit empties into a dish. The turntable and the pumps as well as the control valves are controlled by a computer program (by software) in such a manner that an amount or a multiple of a given base amount fixed in the recipe is transported by the pumps into the dish. After the amounts fixed by the recipe and corresponding in their composition to the desired tone of the hair dye or intensive toner have been dosed into a dish, the amount in the dish is mixed.

For example, 11 magazines with 11 bags are placed on the motor-driven turntable of the device for producing classic hair dyes and intensive toners in accordance with the described method. Of the 11 bags, 7 bags (bags 1 to 7) are filled with base components, 2 bags (bags 8, 9) with alkalizing agent, 1 bag (bag 10) with carrier mass and 1 bag (bag 11) with peroxide. Over 500 color shadings can be produced both as classic ammonia-containing hair dye and as intensive toner with such provisions by means of the device and the entered recipes in as far as the method of the invention is followed, that is, the base components filled into the bags receive only as much alkalizing agent, e.g. sodium hydroxide in a precursor stage as is necessary for the dissolving and stabilizing of the intermediates (developer and coupler), the direct dyes and the carrier mass. More alkalizing agent, depending on the type of coloring, is added only after the mixing of the base components, that is, after the base components have been placed into a dish or a container in accordance with the recipe. As a result of this two-stage method both ammonia (in the case of the classic hair dye) and so-called substitutes such as, e.g., ethanolamine, AMP, etc. (in the case of the intensive toner) can be added. The individual intermediates (developer and coupler), the direct dyes and the associated substances are dissolved in the precursor stage in order to produce the individual base components at 70 to 80° C. in the aqueous phase. The alkalizing agent is subsequently added together with the fatty phase and emulsified. Finally, treatment substances and perfume substances are mixed in during the cooling-off process at approximately 50° C.

When selecting a tone, e.g., by using samples on a color card, the computer can calculate, after the tone has been entered, e.g., as a code, the individual requirement for the desired color amount based on the recipe in the computer and/or on other selected parameters, and dose this amount into the dish.

This type of production makes a very rapid creating of classic, ammonia-containing hair dyes as well as of intensive toners possible for the hairdresser or the customer, based on the recipes stored in the memory.

The base components filled into the bags are composed of a liquid, gelatinous or creamy carrier mass with treatment substances, a coupler, a developer, a direct dye (direct dyes) as well as of the amount of an alkalizing agent necessary for dissolution and stabilization.

The following are used as developer:

p-touylenediamine [sic; p-toluylenediamine] 0.1 to 20%, p-phenylenediamine 0.1 to 5%, p-aminophenol, 0.1 to 2%, 2-chloro-p-phenylenediamine sulfate 0.1 to 5%, 4-amino-3-methylphenol 0.1 to 5%, N,N-bis (2-hydroxyethyl)-p-phenylenediamine sulfate 0.1 to 5%.

The following are used as coupler:

Resorcinol 0.1 to 5%, 4-chlororesorcinol 0.1 to 5%, 2-methylresorcinol 0.1 to 5%, 1-naphthol 0.1 to 1%, m-aminophenol 0.1 to 5%, p-amino-o-cresol 0.1 to 5%, 5-amino-6-chloro-o-cresol 0.1 to 5%, 2-methyl-5-hydroxyethylaminophenol 0.1 to 5%, 2,4-diaminophenoxyethanol (HCl or H2SO4) 0.1 to 5%, 1,5-dihydroxynaphthalene 0.1 to 3%, 1,6 dihydroxynaphthalene 0.1 to 3%, 2,6-diaminopyridine 0.1 to 3%, 2-amino-4-hydroxyethylaminoanisole sulfate 0.1 to 5%, 2-amino-3-hydroxypyridine 0.1 to 5%.

The following are used as direct dye:

4-nitro-o-phenytenediamine 0.1 to 5%, 2-nitro-p-phenylenediamine 0.1 to 5%, 6-chloro-4-nitro-2-aminophenol 0.1 to 10%, sodium picramate 0.1 to 2%, picramic acid 0.1 to 2%, 4-amino-3-nitrophenol 0.1 to 4%, 4-hydroxypropylamino-3-nitrophenol 0.1 to 5%, 3-nitro-p-hydroyethylaminophenol [sic; 3-nitro-p-hydroxyethylaminophenot] 0.1 to 5%, HC red 3 and 13, each 0.1 to 5%, HC yellow 2, 4 and 5, each 0.1 to 5%, HC blue 2 0.1 to 5%, basic red 2, 22, 46, 51 and 76, each 0.1 to 4%, basic blue 3, 7, 9, 26, 47 and 99, each 0.1 to 3%, basic yellow 11, 28 and 57, each 0.1 to 3%, basic brown 4, 16 and 17, each 0.1 to 3%, basic violet 4 and 14, each 0.1 to 3%, disperse violet 1 0.1 to 3%.

The following are used as active treatment substances:

Panthenol 0.1 to 5%, allantoin 0.1 to 0.5%, synthetic oils 1 to 5%, silicones 1 to 5%, vegetable oils such as jojoba oil, wheat-germ oil, corn oil, meadowfoam seed oil, each 1 to 10%, vitamins E—acetate 1 to 15%, UVA- and UVB filters 1 to 5%, silk proteins 1 to 4%, keratin hydrolysate 1 to 4%, collagen hydrolysate 1 to 4%, wheat protein 1 to 4%, elastin hydrolysate 1 to 4%.

The following are used as alkalizing agents:

Ammonium hydroxide 1 to 25%, ethanolamine 1 to 100%, aminomethylpropanol 1 to 100%, sodium hydroxide 1 to 10%, potassium hydroxide 1 to 10%.

The following is used as peroxide:

Hydrogen peroxide 1 to 35%.

The production of mixtures will be explained in detail using a few examples for a better understanding. The 11 bags based on a device provided with 11 bags in accordance with the previously described contents consequently contain the previously described filling contents. This concerns seven base components, two alkalizing agents and one bag with peroxide and one with pure carrier mass (see Table 1). It is of course just as possible to use another number of bags with other base components or another composition. The computer program and the recipes would then have to be correspondingly altered. The example with eleven bags is intended only to explain in detail the use of the base components for different tones and for the use as classic, ammonia-containing hair dye or as intensive toner without ammonia.

The following cream carrier mass 018 (without developer, coupler, direct dye) serves by way of example as a creamy carrier mass with treatment substances in which carrier mass the intermediates (developer and coupler) can be dissolved individually and in certain concentrations that can also differ if necessary:

| | |
|---|---|
| Oleic acid | 2.00% |
| Oleth-20 | 3.60% |
| Cetearyl alcohol | 15.00% |
| Sodium hydroxide 10% | 1.50% |
| EDTA | 0.10% |
| Sodium lauryl sulfate | 0.50% |
| Sodium sulfite | 0.40% |
| Ascorbic acid | 0.10% |
| Perfume | 0.30% |
| Hydrolyzed keratin | 0.50% |
| Aqua | qs |

Any desired color shadings can be composed with base components based on this carrier mass which can be obtained, depending on the type of coloring, with ammonia and/or other alkalizing agents The examples cited in the following Table 1 show that classic hair dyes with ammonia or intensive toners can be produced in accordance with the described production method depending on the recipe and the alkalizing agents used (bags 9 and 10).

TABLE 1

| | | Intensive toners without | | | | | |
|---|---|---|---|---|---|---|---|
| | | ammonia | | | Hair dyes with ammonia | | |
| Bag no. | Bag contents | Blond | Violet | Red | Blond | Violet | Red |

Stage 1

| | | | | | | | |
|---|---|---|---|---|---|---|---|
| Bag 1 | p-toluylenediamine 5% | 0.75 ml | 1.65 ml | 0.50 ml | 0.75 ml | 1.65 ml | 0.50 ml |
| | in carrier mass 018 | | | | | | |
| Bag 2 | p-aminophenol 1% | 0.55 ml | 2.15 ml | 3.30 ml | 0.55 ml | 2.15 ml | 3.30 ml |
| | in carrier mass 018 | | | | | | |
| Bag 3 | p-amino-o-cresol 2% | — | 1.45 ml | 0.85 ml | — | 1.45 ml | 0.85 ml |
| | in carrier mass 018 | | | | | | |
| Bag 4 | resorcin 2% | 0.60 ml | — | — | 0.60 ml | — | — |
| | in carrier mass 018 | | | | | | |
| Bag 5 | m-aminophenol 1% | 0.15 ml | 0.45 ml | — | 0.15 ml | 0.45 ml | — |
| | in carrier mass 018 | | | | | | |
| Bag 6 | 1-naphthol 1% | — | 0.50 ml | 2.00 ml | — | 0.50 ml | 2.00 ml |
| | in carrier mass 018 | | | | | | |
| Bag 7 | HC red #3 1% | — | — | 0.35 ml | — | — | 0.35 ml |
| | in carrier mass 018 | | | | | | |
| Bag 8 | carrier mass 018 | 4.50 ml | 0.80 ml | — | 4.50 ml | 0.80 ml | — |

Stage 2

| | | | | | | | |
|---|---|---|---|---|---|---|---|
| Bag 9 | ethanolamine 10% | 3.00 ml | 3.00 ml | 3.00 ml | — | — | — |
| | in carrier mass 018 | | | | | | |
| Bag 10 | ammonium hydroxide | — | — | — | 3.00 ml | 3.00 ml | 3.00 ml |
| | 6% | | | | | | |
| | in carrier mass 018 | | | | | | |

Stage 3

| | | | | | | | |
|---|---|---|---|---|---|---|---|
| Bag 11 | cream peroxide 6% | 10.00 ml | 10.00 ml | 10.00 ml | 10.00 ml | 10.00 ml | 10.00 ml |

| | | | | | | |
|---|---|---|---|---|---|---|
| Total ready to-use dye mass | 20.00 ml | 20.00 ml | 20.00 ml | 20.00 ml | 20.00 ml | 20.00 ml |

The examples in Table 1 show that not only 3 different color tones can be produced with 11 bags by altering the composition and/or amounts but also that these color tones are available both as classic, ammonia-containing hair dye as well as ammonia-free intensive toner in as far as the mixing of the bag contents 1 to 8 takes place in a precursor stage and only thereafter are the contents of bag 9 or 10 mixed in as alkalizing agent. Of course, as previously explained, more color tones can be produced by other compositions, in which case other recipes must be used. It is advantageous if the ratio of peroxide and base components is 50:50. However, it is absolutely possible to alter this ratio.

On the other hand, the sellers in the market divide the dyes into two separate areas—on the one hand, the area of classic, ammonia-containing hair dyes that simultaneously brighten the hair during the dyeing process and, on the other hand, intensive toners that permit a tone-in-tone dying without brightening to be carried out. These two coloring systems are obligatorily produced and marketed individually. As a rule the classic hair dye assortment has approximately two to three times as many shades as the intensive toners have, partly because the space available to the hairdresser is limited.

According to the invention a hairdresser or shopkeeper can now offer an almost unlimited assortment of classic, ammonia-free hair dyes as well as an intensive toning with only one to two additional bags (see Table 1). The described method is particularly advantageous during on-site production, that is, in the salon or shop. It is thus associated not only with a significant reduction of storage space but also the great number of color tones is almost the same for both types of hair dyes.

1. A method for producing hair dyes and/or intensive toners in which the intermediates (developer and coupler) and the direct dyes are dissolved in liquid or creamy carrier masses that are mixed together by a computer-controlled device based on recipes stored in the computer to a tone mixture on site, e.g., in a hairdressing salon or shop, characterized in that
the intermediates used (developer and coupler) and the direct dyes are dissolved individually in a precursor stage in a carrier mass with only as much alkalizing agent being added as is necessary for the dissolving and stabilizing of the intermediates (developer and coupler), of the direct dyes and of the carrier mass (called base components for short),
in a further step after the mixing together of the individual base components to a tone in a container, ammonia or appropriate substitutes are selectively added.

2. The method according to claim 1, characterized in that the peroxide is added to the ready-to-use mixture in a further method step.

3. The method according to claim 1 and/or 2, characterized in that 1 to 25% ammonium hydroxide or 1 to 100% ethanolamine or 1 to 100% aminomethylpropanol or 1 to 10% sodium hydroxide or 1 to 10% potassium hydroxide is used as alkalizing agent.

4. The method according to claim 1, characterized in that the intermediates (developer and coupler) and the direct dyes are dissolved with the associated substances at 70 to 80° C. in the precursor stage in order to produce a base component in an aqueous phase, that the alkalizing agent is subsequently added together with the fatty phase and emulsified, and that during the cooling-off process the treatment substances and perfume substances are mixed in at approximately 50° C.

5. The method according to claim 4, characterized in that the base components are filled at 45 to 40° C. into suitable containers, e.g., flexible bags suitable for dosing the base components in a device provided to this end.

6. A device for carrying out the method according to claim 1 for producing tone mixtures, characterized in that the bags filled with base components are dosed in a computer-controlled manner into a container or catch dish, which base components are first combined on site, e.g. in a hairdressing salon or in a shop, that the desired alkalizing agent or agents are added in a subsequent step as required, and that the appropriate amount of peroxide is subsequently added at this point when used in a salon, and for use at home the peroxide amount is filled separately into a container and given to the user.

7. The device according to claim 6, characterized in that the base components are present in various concentrations stored in the storage device of the dosing device.

8. The device according to claim 6, characterized in that the peroxides are present in various concentrations stored in the storage device of the dosing device.
